(19) United States
(12) Patent Application Publication
(10) Pub. No.: US 2015/0297472 A1
(43) Pub. Date: Oct. 22, 2015

(54) DEVICES AND METHODS FOR DELIVERY OF ORAL TREATMENTS

(71) Applicant: H2X, Inc., Tucson, AZ (US)

(72) Inventors: Vincent J. Marold, Tucson, AZ (US); Brian Midei, Tucson, AZ (US)

(21) Appl. No.: 14/755,614

(22) Filed: Jun. 30, 2015

Related U.S. Application Data
(63) Continuation of application No. PCT/US2013/078261, filed on Dec. 30, 2013.
Application No. PCT/US2013/078261 is a continuation-in-part of application No. 14/743,277, filed on Jun. 18, 2015.
Application No. 14/743,277 is a continuation of application No. 14/139,224, filed on Dec. 23, 2013, now Pat. No. 9,060,833.
Application No. 14/139,224 is a continuation of application No. 13/789,198, filed on Mar. 7, 2013, now abandoned.
Application No. 13/789,198 is a continuation of application No. PCT/US2012/028140, filed on Mar. 7, 2013.
Application No. PCT/US2012/028140 is a continuation-in-part of application No. 14/743,249, filed on Jun. 18, 2015.
Application No. 14/743,249 is a continuation of application No. 14/139,224, filed on Dec. 23, 2013, now Pat. No. 9,060,833.
Application No. 14/139,224 is a continuation of application No. 13/789,198, filed on Mar. 7, 2013, now abandoned.
Application No. 13/789,198 is a continuation of application No. PCT/US2012/028140, filed on Mar. 7, 2012.
(60) Provisional application No. 61/748,020, filed on Dec. 31, 2012.

(51) Int. Cl. A61K 8/02; A61K 9/00; A61K 31/522; A61K 36/28; A61K 31/465

(52) U.S. Cl. CPC A61K 8/0229; A61K 36/28; A61K 31/465; A61K 31/522; A61K 9/0053; A61K 2800/92

(57) ABSTRACT

A device for delivering an oral treatment and a method for preparing such, the product exhibiting improved capacity for delivering oral additives to the user. The device may be prepared by pretreating a wooden substrate to increase porosity and decrease hardness. The device is then immersed in a compound comprising the oral additive, a masking agent, and/or a sweetening agent, where the substrate absorbs the compound. The resulting device provides a cost-effective method for prolonging a user's exposure to the desired oral additive.

### BACKGROUND

The present technology relates generally to devices, in particular dental implements, including flavored toothpicks, and methods for making the same.

Toothpicks have been used as dental implements by man for centuries. Numerous studies in recent history have demonstrated that wood toothpicks are comparable to other dental implements and devices at cleaning teeth effectively. In fact, wood toothpicks are much easier to use than dental floss and are thus much more likely to be used by children and the elderly to promote dental health. Further, the use of toothpicks may facilitate the overcoming of oral fixations such as smoking, over-eating, and the like.

Toothpicks are typically formed as slivers of material, such as wood, having at least one end that is pointed for inserting in between the user's teeth. There are several methods currently available by which a toothpick can be manufactured to include other features, such as a flavoring or helpful additive, such as fluoride. These methods typically involve spray coating, pan coating or soaking the toothpick in a flavored compound or oil. However, these products generally lose their flavor and the effectiveness of any other additives in a relatively short time frame. Moreover, such methods are often less than satisfactory at sufficiently infusing wood dental implements with a desired amount of active ingredient, and often provide diminishing effect over multiple uses.

Accordingly, there remains a need to produce dental implements with a greater ability to hold and to effectively deliver active ingredients.

### BRIEF SUMMARY

In certain embodiments, the present technology is directed to a device for oral delivery of an oral treatment, the device comprising a substrate of unitary construction, the substrate being formed of a porous material that has been pretreated in a pretreatment wash to increase the porosity and decrease the hardness of the substrate, wherein the substrate is infused with a compound containing an oral additive.

In other embodiments, the present technology is directed to a method of preparing a device for oral delivery of an oral treatment, the device being formed of a porous substrate of unitary construction, the method comprising the steps of:

pretreating the porous substrate in a pretreatment wash at a raised temperature to increase the porosity of the substrate;

preparing a compound comprising an oral additive;

contacting the porous substrate with the compound at a raised temperature for a predetermined length of time; and

curing the device in a low humidity environment.

In other embodiments, the present technology is directed to method for preparing a device for treating an oral ailment, comprising the steps of:

preparing a compound comprising an oral additive;

exposing a porous substrate to the compound within a sealed chamber;

subjecting the sealed chamber to a vacuum pressure for a period of time; and

removing the porous substrate to an environment of low relative humidity for curing.

### BRIEF DESCRIPTION OF THE DRAWINGS

FIG. 1 is a schematic displaying a method for treating a substrate according to a first aspect of the present disclosure;

FIG. 2 is a flowchart displaying a method of preparing a device in accordance with another aspect of the present disclosure; and

FIG. 3 is a flowchart displaying a method of preparing a device using vacuum pressure in accordance with another aspect of the present disclosure.

### DETAILED DESCRIPTION

As used herein, unless otherwise indicated, all percentages are by weight.

The present disclosure provides, in certain embodiments, a device such as a dental implement and a method for preparing such device, the product fulfilling the need for a greater capacity and effectiveness in delivering flavoring and other desired additives. Other advantages in performance will be apparent to one having ordinary skill in the art.

In certain embodiments, the present technology is directed to a device for delivering an oral treatment, formed as a single piece of porous substrate, such as from a wood material. The substrate is infused with a compound containing the desired oral additive, along with other flavoring agents, masking agents, or sweeteners, if desired.

The substrate may be pretreated to achieve an increase in porosity and a decrease in hardness. In certain embodiments, the pretreatment is performed with a pretreatment wash of warm water, which may also include other chemicals to aid in the process, such as sodium hydroxide (NaOH). Through the pretreatment, the hardness of the toothpick is typically decreased by about 10 to about 20%.

A second aspect of the present disclosure provides a method of preparing a device for delivery of an oral treatment, for example, for treating an oral ailment, where the device is placed in contact with a compound, containing an oral additive, at a raised temperature before curing the toothpick in an environment of low humidity. In certain embodiments, the device may be placed in a pretreatment wash as described above.

Yet another aspect of the disclosure provides a method for preparing a device for treatment of an oral ailment, wherein a compound containing an oral additive is infused into a substrate by placing the substrate in a sealed chamber in contact with the compound under a vacuum. The substrate may then be cured in a low humidity environment with heated, dehumidified air.

In the present disclosure, reference is made to the accompanying drawings, which form a part hereof, and in which is shown, by way of illustration, various embodiments of the present technology. It is understood that other embodiments may be utilized and changes may be made without departing from the scope of the present invention.

In a first aspect, the present disclosure provides an improved device, for example, a flavored dental implement, such as, but not limited to, a toothpick. The device may be made from any number of substrates, but in various embodiments these substrates may include wood, for example, any porous hardwood including but not limited to birch (e.g., yellow birch, white birch, black birch or any other hardwood tree of the genus Betula) maple, oak or walnut or the like.

As used herein, the term “dental implement” means any structure that can be inserted into the mouth of a user, including a device that can contact the gums, teeth, tongue or other oral mucosa of the user, to provide a benefit to the user.

As used herein, the term “oral treatment” means any effect that is desired to be delivered to a user's mouth, tongue, gums, oral mucosa, teeth or other internal surfaces of the mouth or throat, including but not limited to an oral ailment.

As used herein, the term “oral additive” means any agent or composition that a user may desire to deliver to his or her mouth or throat for any benefit or purpose, including but not limited to a flavoring agent or any composition that improves the oral or general health of a user; for example, an anticaries agent, antibacterial agent, analgesic agent, or agent that stimulates salivation or otherwise combats dry mouth, a common condition also known as xerostomia. In certain embodiments, the dental implements and methods discussed herein comprise birch wood, which is the most common toothpick material used in North America.

Compared to conventional toothpicks, the devices (such as dental implements) of the present disclosure are softer and will not easily damage the dentin or enamel and teeth; further, they are able to absorb a larger amount of flavoring and other desired additives than those currently known. In certain embodiments, these improved characteristics are the direct result of a pretreatment of the substrate prior to contact with the flavoring. In certain embodiments, where the substrate is wood, the pretreatment of the wood removes certain constituents of the wood structure to bring about the softer feel and increased porosity. The increased porosity allows the device to absorb an increased amount of flavoring or other additives. The increased softness is appealing to users because it provides for a more gentle experience, reducing the risk of injuring the user's gums and oral mucosa. In certain embodiments, further improved characteristics are realized through the application of a vacuum step to the devices, as will be discussed later.

### Pretreatment Wash

In certain embodiments, the devices of the present technology are pretreated with a pretreatment solution before contact with the additive. Such solution may be water-based or alcohol-based, depending on the characteristics of the additive and flavoring considerations. In various embodiments, the solution is kept at an elevated (raised) temperature, for example, about 140 to about 175° F., about 145 to about 175° F., about 148 to about 165° F. or about 150 to about 160° F. As used herein, “raised temperature” means a temperature higher than room temperature. These ranges have been found to be optimal; the elevated temperature should not be so high so as to warp or otherwise damage the substrate. The length of time that the solution remains at the elevated temperature is also dependent upon the characteristics of the additive and other constituents.

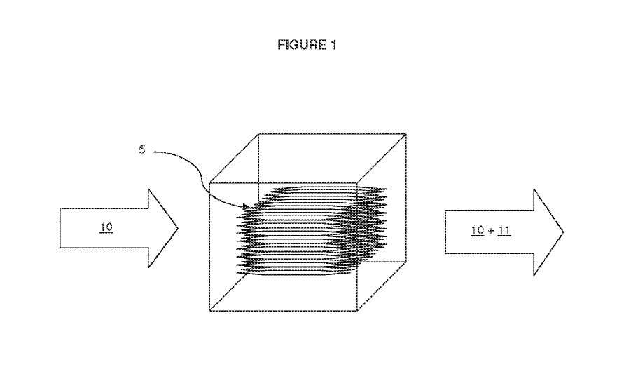

Referring to FIG. 1, in certain embodiments the wood is pretreated by placing a batch of previously manufactured devices 5 in a wash 10 of warm water. In certain embodiments, the water should be flowing, or at least agitated, in order to open up the pores in the wood, or carry away wood debris, “heavy cellulose” and other constituents 11 of the wood.

In one exemplary embodiment, a batch of devices may be placed in a pretreatment wash of water at a temperature of about 150 to about 160° F. for about 2 minutes to about 1 hour, or about 5 minutes to about 45 minutes, or about 5 minutes to about 30 minutes, or about 5 minutes to about 15 minutes. After this pretreatment step, in certain embodiments the birch wood is estimated to have an increased porosity of over 30%, as observed by the additional uptake of injected material. In various embodiments, the devices may comprise other materials, e.g., other types of wood or substrates, and after treatment may exhibit increased porosity of about 10% or greater, about 15% or greater, or about 20% or greater than devices that have not been pretreated.

In order to decrease the pretreatment time or temperature, or to target specific constituents of a particular substrate, the pretreatment wash may also include other chemically active substances. For example, the pH of the wash may be slightly basic by adding a dilute solution of sodium hydroxide (NaOH) or sodium bicarbonate to result in a solution with pH above 7 (in various embodiments, about 7.5 to about 10, about 7.5 to about 10, or about 7.5 to about 9.5). In certain embodiments, this will target the cellulose structure of wood. In such embodiments, a basic solution may be advantageous because of its potential to remove portions of the cellulose, thereby increasing the porosity, without otherwise compromising the structural integrity of the wood. However, in certain embodiments, acidic solutions may also be used with wood to remove other constituents; by, for example, addition of an acid or other agent that results in a solution with a pH below 7 (in various embodiments, about 5 to about 6.5 or about 5.5 to about 6). Other chemical specific solutions may also be used with wood or other substrates as necessary or desired.

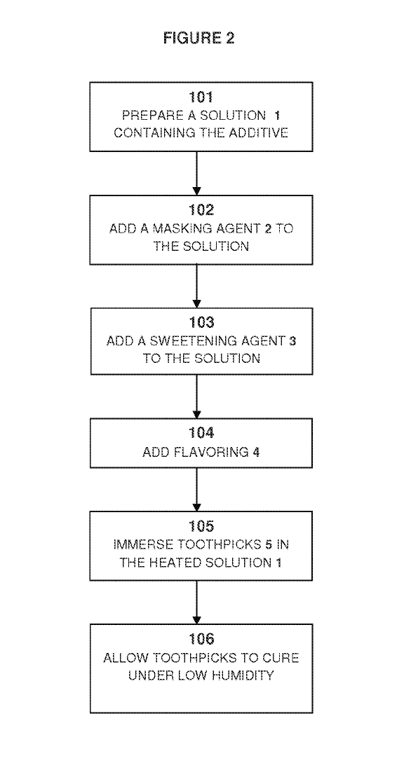

In another aspect, the present disclosure provides a process for making the flavored devices described above. With reference to FIG. 2, a device in accordance with the present technology can be made by first preparing a super-saturated solution 1 of the desired additive (step 101). In a non-limiting example, a super-saturated caffeine solution can be prepared by heating water to an elevated temperature sufficient to dissolve caffeine, for example in the range of about 140 to about 180° F. Once the desired additive has been dissolved, a masking agent 2 is added to the solution to mask any undesired flavor characteristics of the additive (step 102). A sweetening agent 3 is added (step 103), and a flavoring 4 is added (step 104). In certain embodiments, these steps may be in any order that is, the embodiments herein are not limited in whether either or both of the masking agent or sweetening agent are added before or after the additive

In certain embodiments, the desired oral additive may comprise one or more of a number of biologically active compounds suitable for ingestion and having a variety of advantageous features. For example, in certain embodiments, such biologically active compounds may include a variety of botanicals, vitamins, homeopathic compounds, synthetic compounds, and the like, such as are now known-or come to be known-as suitable additives or nutritional supplements.

In certain embodiments, the oral additive may comprise an agent that targets and treats a particular medical condition—for example, heart disease, diabetes, high blood pressure, infection by viruses, bacteria or any other microorganisms, or any condition for which application of an orally-ingestible agent or agent that can be placed sublingually can be useful. For example, in certain embodiments, an oral additive herein may comprise nitroglycerin, and a user with a heart condition may be able to use such a device to deliver this oral additive as an alternative to taking a pill. Similarly, an oral additive herein may comprise an agent for treating conditions related to diabetes, high or low blood pressure, asthma, allergies, epilepsy, a viral, bacterial or yeast infection, or any psychiatric or neurological condition for which rapid ingestion of a suitable treatment agent may be advantageous or desirable.

In certain embodiments, the oral additive may comprise an anticaries agent, an anesthetic or analgesic agent. For example, suitable anticaries agents may include, but are not limited to, fluoride, phosphorous containing agents (including sodium monofluorophosphate and calcium glycerophosphate and the like), clorhexidine, antimicrobials, natural extracts and metals. For example, suitable anesthetic agents may include, but are not limited to, local anesthetics such as lidocaine, benzocaine or any other anesthetics ending in “-caine,” or any agents that can be applied topically to the mouth and throat and that provide numbing. For example, suitable analgesic agents may include, but are not limited to, natural or synthetic painkillers such as non-steroidal anti-inflammatory drugs (NSAIDs) such as ibuprofen, acetaminophen, aspirin, naproxen, paracetamol, cyclooxygenase (COX-1 and COX-2) inhibitors, opiates, and any other natural or synthetic agents that inhibit the sensation of pain.

In certain embodiments, the oral additive may comprise a stimulant, e.g., a composition for increasing energy, reducing lethargy, reducing sleepiness, decreasing appetite or increasing concentration. In certain embodiments, the stimulant may be caffeine or nicotine, and the device herein may be useful as an addiction recovery aid.

In certain embodiments, the oral additive may be a compound used in the treatment of oral ailments. As used herein, the term “oral ailment” means any of a variety of conditions experienced by users for which the devices of the present technology may be used to treat or ameliorate the symptoms, or decrease discomfort to a user. Examples of oral ailments that may be treated with the devices herein include any disorder of the mouth, tongue, gums, oral mucosa, teeth or other internal surfaces of the mouth or throat, such as, for example, caries (cavities), dry mouth, suppressed salivation, oral infection, gum disease, canker sores or other oral or throat trauma, pain or discomfort.

One example of such an application is the use of spilanthol and its derivatives as an oral additive, including various formulations comprising essential oils obtained from Spilanthes oleracia Jacquin, Spilanthes acmella, or the like. These and other substances have a benefit in treating oral ailments such as, for example, dry mouth. As used herein, “treating” or “treatment” refers to amelioration or reduction of the effects of an ailment or condition by any amount.

Xerostomia, or dry mouth, results from numerous diseases and causes including but not limited to: nasal congestion from seasonal or chronic allergies, asthma, sinus infection, hormone changes, medications, illness, mouth breathing, chronic or acute stress, extended talking, gastric acid reflux, bulimia, chemotherapy or long term illness, poor diet with lack of minerals or vitamins, fear, depression, dehydration, aging, decreased ability to chew, age, oxygen deprivation, and use of hospital feeding tubes.

Symptoms of dry mouth may include: decrease saliva that may be foamy, thick or ropy, a dry fissured tongue, Candidiasis formation, difficulty eating foods or with taste, persistent denture troubles, difficulty swallowing, sensitivity to acidic, salty and spicy foods, and extensive and sometimes rampant dental decay, especially in areas not usually prone to decay.

Dry mouth often results in an environment that is acidic and can damage the associated tooth structures. Diseases such as Parkinson, Sjogren's Syndrome, diabetes and AIDS, as well as bone marrow transplants, can also cause dry mouth. Often, many diseases require medications that subsequently lead to dry mouth or compound its effects.

Acidic pH in the mouth of 5.5 or below causes demineralization of tooth structure and can initiate or continue to grow dental caries. Increasing salivation with appropriate Sialogogues can counteract these effects by diluting and neutralizing the acid content in the mouth. Thus, the various agents discussed herein that treat dry mouth can also be characterized as anticaries agents.

When used as an additive, spilanthol delivers a pleasant taste and a mild anesthetic effect, the result of which often causes the user to produce saliva. Some of the benefits of spilanthol and other substances, as well as various theories on the formulation of such compounds, are discussed in U.S. Pat. No. 3,720,762 to Hatasa et al., the contents of which are incorporated herein by reference.

Other compounds for treating oral hygiene and related ailments, including compositions based on Hellopsis longpipes root, are discussed in European Patent Publication No. EP1466003 A1, the contents of which are also incorporated herein by reference. Oral health benefits may also be obtained from other plant extracts, including Centella plant extracts, Jatropha plant extracts, Aegle plant extracts, Terminalia plant extracts, Phyllanthus plant extracts, and Bacopa plant extracts, as well as Spilanthes plant extracts. These plant extracts, and variations thereof, are discussed in U.S. Patent Publication No. 2012/0027697, the contents of which are incorporated herein by reference.

By using the devices of the present technology as a delivery vehicle for an additive with oral benefits, such as spilanthol, the user is able to receive a prolonged exposure to the desired additive when compared with other delivery methods such as oral rinses or medicated chewing gum. Often, the prolonged exposure to the oral additive is desirable to maximize the period of relief received from the additive or to otherwise increase the health benefits due to the prolonged exposure. This is also true for additives used to treat addictions, such as smoking cessation additives, whereby the user is able to suppress the desire for the addictive substance for a longer period of time.

Treatment with Additive

As can be seen in the Figures, in an illustrative embodiment, whether or not pretreatment of the devices has occurred, the devices 5 are added to a heated solution comprising the additive, and allowed to soak therein for a period of time sufficient for the solution to be fully absorbed into the toothpick (step 105). In certain embodiments, this time can be about 1 hour to about 5 hours, about 1 hour to about 4 hours, about 1 hour to about 3 hours or about 1 hour to about 2 hours. For example, the devices may be added to the heated solution and left to soak therein for the determined period of time.

In certain embodiments, the additive comprises an agent that ameliorates dry mouth, including but not limited to spilanthes. In various embodiments, the additive solution comprises about 0.5 to about 5%, about 0.75 to about 4.5%, about 1 to about 4%, about 1.5 to about 3.5% or about 2 to about 3% by weight of spilanthes.

In this exemplary embodiment, the devices, once infused with the additive, are then removed from the solution, drained and allowed to dry under conditions of low relative humidity 106. In certain embodiments, the low humidity environment is less than about 18% relative humidity, less than about 15% relative humidity, less than about 12% humidity or about 8 to about 12% relative humidity. In certain embodiments, to expedite the drying process, dehumidified air, heated air, or a combination thereof may be used to increase the rate at which the picks give up the moisture contained therein. By way of example, in certain embodiments the devices are placed into a chamber, and dehumidified air may be alternately dehumidified into or drawn from the chamber. Using this method, the devices should be sufficiently cured within about one hour.

In certain embodiments, the solution containing the oral additive will comprise about 50 to about 70% water, about 15 to about 25% flavoring agent, about 1 to about 3% masking agent, about 1 to about 3% sweetener (powder), and about 7 to about 10% of the oral additive, for example, caffeine (anhydrous). In various embodiments, these amounts may vary depending on the additive, the form it is provided in, and the base of the solution (water, alcohol, or otherwise). For instance, the artificial sweetener need not be provided in powder form, but could instead be provided as a liquid, and thereby comprise a greater volume percentage of the overall solution.

In certain embodiments, the oral additive may comprise a flavoring agent. The flavoring agent may be a natural or artificial flavor. For example, the flavoring agent may be a water-soluble flavoring agent and may comprise one or more constituents, including, for example: almond flavor, almond toffee flavor, amaretto flavor, apple flavor, apple pie flavor, apricot creme flavor, bailey's Irish cream flavor, baklava flavor, banana flavor, banana cream flavor, banana nut bread flavor, bananas foster flavor, almond biscotti flavor, chocolate biscotti flavor, lemon & icing biscotti flavor, vanilla nut biscotti flavor, brown sugar flavor, black cherry flavor, triple berry flavor, bourbon flavor, Butterfinger™ flavor, butter cream flavor, butter pecan flavor, butter rum flavor, butterscotch flavor, caramel flavor, caramel apple flavor, caramel latte flavor, caramel macchiato flavor, chai flavor, cherry flavor, chocolate flavor, chocolate cream flavor, chocolate mint flavor, chocolate raspberry flavor, cinnamon flavor, coconut flavor, coconut creme flavor, coconut & rum flavor, cranberry flavor, crème brulee flavor, creme de menthe flavor, dulce de leche flavor, egg nog flavor, English toffee flavor, espresso flavor, frangelica flavor, french vanilla flavor, green tea flavor, hazelnut flavor, grand marnier flavor, highland grogg flavor, honey flavor, Irish cream flavor, Kona flavor, lemon drops flavor, licorice flavor, lime flavor, macadamia nut flavor, mandarin orange flavor, mango flavor, margarita flavor, marshmallow flavor, mocha flavor, passion fruit flavor, peach flavor, peaches and cream flavor, pear flavor, peppermint flavor, pineapple flavor, piña colada flavor, pistachio flavor, pomegranate flavor, praline flavor, pumpkin pie flavor, rain forest crunch flavor, raspberry flavor, rose flavor, rum flavor, Santa's Xmas flavor, Snickers™ flavor, snickerdoodle flavor, swiss chocolate almond flavor, spice flavor, strawberry flavor, oil-based sweetener flavor, sweet potato pie flavor, Tahitian vanilla flavor, tangerine flavor, tiramisu flavor, toasted almond flavor, toasted coconut flavor, vanilla flavor, vanilla spiced rum flavor, viennese flavor and white chocolate flavor, as well as any currently known flavor or new flavor not yet known.

The sweetening agent may be any suitable natural or artificial sweetener. In some examples, the sweetening agent is an artificial sweetener, selected from the group consisting of sucralose, aspartame, saccharin and acesulfame potassium.

The masking agent may be any suitable masking agent known in the art which is used in pharmaceutical supplements, foods and drinks to mask bitterness and/or enhance flavors. In a non-limiting example, the masking agent is thaumatin or a derivative thereof. Thaumatin is a low calorie flavor modifier comprised of a natural protein extracted from the katemfe fruit, Thaumatococcus daniellii.

### Vacuum Step

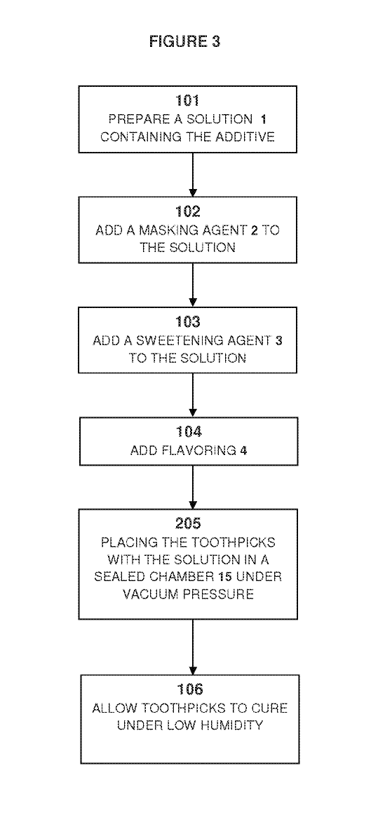

Referring to FIG. 3, in certain examples, vacuum pressure is applied to infuse, or to improve the infusion of, the oral additive(s), such as the flavoring, into the porous substrate of the toothpick (step 205). For purposes of the present disclosure, the word “infuse” refers specifically to the use of vacuum pressure to draw the flavoring and/or the additive solution into the device to fill the porous structure of the substrate (in the case of a porous substrate such as wood). Using this process, it is possible to achieve a nearly homogeneous distribution of additives and/or flavoring throughout the substrate. As used herein, “nearly homogeneous distribution” means that the additives or flavoring are substantially uniformly distributed throughout the substrate, such that a user may consistently experience the effects of such additives or flavorings regardless of which portion of the device is contacted with the oral surfaces.

In certain embodiments, the vacuum step increases the porosity of the substrate by providing a larger surface area for the oral additive to penetrate into the device. In certain embodiments, it has been found that without the vacuum step about 5 to about 7 mg of oral additive can be infused into the device; but with the vacuum step, the amount increases to as much as about 18 to about 20 mg. In certain embodiments, the vacuum step decreases the hardness of the device by about 30 to about 50%, or even by more than about 50% (percentages based on a completely untreated device that has not been pretreated or subjected to the vacuum step).

By way of example, a batch of devices may be placed in a bath of the additive solution in a sealed chamber 15. A vacuum is applied to the chamber, for example, using a mechanical pump or compressor. Other machinery, such as liquid ring vacuum pumps, may also be used, with varying amounts of pressure depending on the size of the batch, the strength of the material, and the solution.

While a small amount of pressure may be effective to significantly increase the uptake of the additive/flavoring, in certain embodiments, application of a vacuum of more than about 28 mmHg is useful to ensure that the substrate is fully saturated with the solution prior to the curing step 106. In certain embodiments, a vacuum that is slightly below atmospheric level, for example, about 5 to about 30% below 1 atm (atmospheric pressure), or about 10 to about 15% below 1 atm, or more than about 10% below 1 atm, is sufficient.

In certain embodiments, the vacuum step 205 may be used in connection with the pretreatment of the substrate. That is, the vacuum step need not be made in connection with the contact with the oral additive, but may be made either before, after or during such step, as well as before, after or during the step of contacting the devices with the pretreatment wash. It has been found herein that applying the vacuum step, whether before or after contact with the additive, results in devices that absorb about 100 to about 300% more of the additive and/or flavoring solutions, whether water or alcohol-based. It has also been found herein that applying the vacuum step decreases the hardness of the substrate. For example, while typical birch wood has a hardness of approximately 1260 lbs., as measured using the “Janka” test, the devices in accordance with certain embodiments herein exhibit hardness levels of about 300 to about 350 lbs. In certain embodiments, applying the vacuum step to the devices herein for about 5 to about 10 minutes, whether done before or after contact with the additive, increases porosity of the substrate by about 250% or more, or about 300% or more. In fact, in certain embodiments, while the pretreatment step does increase the porosity and surface area of the devices, the vacuum step provides unexpectedly superior levels of increased surface area deeper into the devices, and permits a highly desirable level of penetration of the oral active into the device.

Devices prepared according to this process may also exhibit other features, including decreased hardness and increased size (girth). Such devices therefore deliver significantly more of the desired additive, and the increased flavoring is long-lasting, having been infused into the substrate.

It should be emphasized that the above-described embodiments of the present device and process, particularly, and “preferred” embodiments, are merely possible examples of implementations and merely set forth a clearer understanding of the principles of the disclosure. Many different aspects of the disclosure described herein may be designed and/or fabricated without departing from the spirit and scope of the disclosure. For example, the above disclosure discusses wood as the substrate from which the toothpick is formed, but several other materials, both organic and man-made are well within the scope of the present disclosure, in accordance with the skill in the art. All these and other such modifications and variations are intended to be included herein within the scope of this disclosure and protected by the following claims. Therefore the scope of the disclosure is not intended to be limited therein.

1. A device for oral delivery of an oral treatment, the device comprising a substrate of unitary construction, the substrate being formed of a porous material that has been pretreated in a pretreatment wash to increase the porosity and decrease the hardness of the substrate, wherein the substrate is infused with a compound containing an oral additive.

2. The device of claim 1, wherein the substrate comprises wood.

3. (canceled)

4. The device of claim 1, wherein the oral additive comprises a composition chosen from a stimulant, an anticaries agent, an analgesic agent or an agent that treats dry mouth.

5. The device of claim 4, wherein the oral additive comprises spilanthes.

6. The device of claim 4, wherein the oral additive is nicotine or caffeine.

7. The device of claim 1, wherein the compound further comprises a masking agent and a sweetener.

8. The device of claim 1, wherein the compound is infused into the substrate so as to approach a nearly homogeneous distribution.

9. A method of preparing a device for oral delivery of an oral treatment, the device being formed of a porous substrate of unitary construction, the method comprising the steps of:
pretreating the porous substrate in a pretreatment wash at a raised temperature to increase the porosity of the substrate;
preparing a compound comprising an oral additive;
contacting the porous substrate with the compound at a raised temperature for a predetermined length of time; and
curing the device in a low humidity environment.

10. (canceled)

11. The method of claim 9, wherein the porous substrate comprises wood.

12. (canceled)

13. A method for preparing a device for treating an oral ailment, comprising the steps of:
preparing a compound comprising an oral additive;
exposing a porous substrate to the compound within a sealed chamber;
subjecting the sealed chamber to a vacuum pressure for a period of time; and
removing the porous substrate to an environment of low relative humidity for curing.

14. The method of claim 13, wherein the porous substrate comprises wood.

15. The method of claim 13, wherein the compound further comprises a masking agent, a sweetening agent or a flavoring.

16. (canceled)

17. (canceled)

18. The method of claim 13, wherein the oral ailment is dry mouth.

19. The method of claim 13, wherein the oral additive comprises a composition chosen from a stimulant, an anticaries agent, an analgesic agent, an anesthetic agent or an agent that treats dry mouth.

20. The method of claim 19, wherein the oral additive is spilanthes.

21. The method of claim 19, wherein the oral additive is nicotine or caffeine.

22. The method of claim 13, wherein the curing step is performed in an environment of less than about 18% relative humidity.

23. The method of claim 12, wherein the vacuum pressure is applied using a mechanical pump.

24. The method of claim 12, wherein the vacuum pressure is more than about 10% below atmospheric pressure.
